(19) United States
(12) Patent Application Publication
(10) Pub. No.: US 2008/0019911 A1
(43) Pub. Date: Jan. 24, 2008

(54) Method for decreasing blood glucose and improving glucose tolerance using angiopoietin-like protein 4

(76) Inventors: Aimin Xu, Baguio Villa (HK); Karen S.L. Lam, Fulham Garden (HK)

Correspondence Address: COOPER & DUNHAM, LLP, 1185 AVENUE OF THE AMERICAS, NEW YORK, NY, 10036, US

(21) Appl. No.: 11/375,297

(22) Filed: Mar. 14, 2006

Related U.S. Application Data
(60) Provisional application No. 60/673,091, filed on Apr. 20, 2005.

(51) Int. Cl. A61K 49/00; A61K 38/16; A61P 3/10; C12Q 1/02; G01N 33/68

(52) U.S. Cl. 424/9.2; 435/29; 436/86; 514/12; 514/789

(57) ABSTRACT

Angiopoietin like protein 4 is a novel circulating protein predominantly expressed in adipose tissue and liver. Several recent studies demonstrated that angiopoietin like protein 4 is the target gene of peroxisome proliferation activators, the agonists of which are widely used as the anti-diabetic and lipid-lowering drugs. The invention provides a method for decreasing blood glucose or improving insulin sensitivity in a mammal in need thereof, comprising administering to the patient an amount of angiopoietin like protein-4 (ANGPTL4) polypeptide effective to decrease blood glucose, to improve glucose tolerance, or to increase insulin sensitivity.

This application claims priority of U.S. Provisional Application No. 60/673,091, filed Apr. 20, 2006, the contents of which are hereby incorporated by reference into this application.

### FIELD OF THE INVENTION

The invention relates to Angiopoietin-like protein 4, and more particularly to methods for regulating glucose metabolism using angiopoietin-like protein 4.

### BACKGROUND OF THE INVENTION

Adipose tissue is now recognized to be an important endocrine organ that secretes a variety of bioactive peptides, known as adipokines (or adipocytokines). Growing evidence suggests that adipokines are critically involved in regulating energy metabolism, systemic insulin sensitivity, cardiovascular tone and immune response (1, 2). Several adipokines, such as tumor necrosis factor (TNF) α, resistin and interleukine 6, play causative roles in the pathogenesis of insulin resistance, type 2 diabetes and thrombotic diseases (1). On the other hand, leptin and adiponectin possess many beneficial functions on energy metabolism and insulin sensitivity. Leptin has long been viewed as an anti-obesity hormone (3), while adiponectin is an insulin-sensitizing adipokine with direct anti-diabetic, anti-atherogenic and anti-inflammatory functions (4-7).

Angiopoietin-like protein 4 (ANGPTL4), also known as PPARγ angiopoietin-related protein (PGAR), fasting induced adipose factor (FIAF) or hepatic fibrinogen/angiopoietin-related protein (HFARP), is a recently identified adipokine that is predominantly expressed in adipose tissue (8-10). Moderate amount of expression is also present in liver and placenta. Mouse ANGPTL4 is composed of an NH2-terminal coiled-coil domain and a carboxyl fibronectin-like motif, a structural organization conserved in both angiopoietins and angiopoietin-like proteins (8). ANGPTL4 was originally identified as the target gene of peroxisome proliferator-activated receptors (PPAR) (8, 9). The agonists of both PPARγ and PPARα could enhance ANGPTL4 expression and also elevate the circulating levels of this protein in human subjects and rodents (11). In addition, the expression of ANGPTL4 is under nutritional control, with its plasma concentration being increased by fasting and decreased by high fat feeding (9).

The metabolic functions of ANGPTL4 are still poorly understood. It has recently been shown that ANGPTL4 treatment acutely increases plasma triglycerides in mice, suggesting it to be a modulator of lipid metabolism (12, 13). However, these studies showed that the effect of ANGPTL4 on plasma triglycerides is transient, and the long-term effects of this protein on energy metabolism and insulin sensitivity remains to be established.

### SUMMARY OF THE INVENTION

The invention provides a method for decreasing blood glucose or improving insulin sensitivity in a mammal in need thereof, comprising administering to the patient an amount of angiopoietin like protein-4 (AGPTL4) polypeptide effective to decrease blood glucose, to improve glucose tolerance, or to increase insulin sensitivity.

The invention further provides a method for monitoring a treatment for diabetes or insulin resistance in a patient comprising monitoring blood levels of ANGPTL4 polypeptide in the patient receiving treatment.

The invention also provides a method of screening for an agent to determine its usefulness for treatment of diabetes or insulin resistance comprising contacting a mammalian cell expressing a ANGPTL4 polypeptide with the agent, and determining if the agent increases ANGPTL4 polypeptide production, wherein an increase in ANGPTL4 polypeptide production is indicative of usefulness for treatment of diabetes or insulin resistance.

### BRIEF DESCRIPTION OF THE DRAWINGS

Further features and advantages of the invention will become apparent upon review of the following detailed description of the preferred embodiments taken in conjunction with following drawing, in which:

FIG. 1 shows expression of ANGPTL4 protein in C57 mice following tail-vein injection with Adv-ANGPTL4. [A] the schematic diagram of mouse ANGPTL4 protein structure. Note that cysteine 76 and 80 are responsible for oligomerization (20). Asp181, 232 and 320 are the three predicted N-glycosylation sites. SS: signal sequence. [B] Western blot analysis of serum proteins using the anti-FLAG monoclonal antibody. 1 μl of serum from mouse at one day before (−1) or different days after tail vein injection with 5×10⁹ p.f.u of Adv-ANGPTL4 was separated by 12% SDS-PAGE and probed with HRP-conjugated anti-FLAG antibody (Sigma-Aldrich).

FIG. 2 illustrates that over-expression of ANGPTL4 causes a sustained decrease of blood glucose levels and improves glucose tolerance in C57 mice. [A] Blood glucose levels of mice at various days after injection with 5×10⁹ p.f.u of Adv-ANGPTL4 or Adv-Luc. [B] Glucose tolerance test at two weeks after treatment. *, P<0.05; **, P<0.01 compared with Adv-Luc treated group (n=6−8).

FIG. 3 illustrates that the potent therapeutic effects of ANGPTL4 on hyperglycemia, hyperinsulinemia and glucose intolerance associated with db/db diabetic mice. [A] Fasted and fed blood glucose levels of db/db diabetic mice at one and two weeks after injection with 5×10⁹ p.f.u of Adv-ANGPTL4 or Adv-Luc; [B and C] show plasma insulin levels and glucose tolerance test at two weeks after treatment respectively. *, P<0.05; **, P<0.01 compared with Adv-Luc treated group (n=7-9).

FIG. 4 illustrates that ANGPTL4 exerts direct inhibitory effects on glucose production in primary rat hepatocytes. [A] Immunoblotting analysis of ANGPTL4 secreted from primary rat hepatocytes. Cells grown in a 12-well plate were infected with Adv-ANGPTL4 or Adv-Luc (50 p.f.u/cell) for a period of 48 hrs. The conditioned culture media from these cells were subjected to immunoblotting analysis using HRP-conjugated anti-FLAG monoclonal antibody. Note that ANGPTL4 in the culture medium is present as both the full-length form (˜70 kDa) and the cleaved COOH-terminal fragment (˜50 kDa). [B] The rates of hepatic glucose output in the absence or presence of different concentrations of insulin. C: Percentage of inhibition of HGO by various concentrations of insulin in cells infected with either Adv-ANGPTL4 or Adv-Luc. *, P<0.05; **, P<0.01 compared with Adv-Luc treated group (n=6−8).

FIG. 5 illustrates the human ANGPTL4 ELISA system. [A] Purification of the recombinant human ANGPTL4 fragment from E. coli as an antigen for antibody production. 30 μg of proteins from bacterial lysate before (lane 1) or after (lane 2) induction with IPTG, or 5 μg of the purified protein (lane 3) was separated by 12% SDS-PAGE and stained with commassie brilliant blue R250. [B] Human ANGPTL4 standard curve made with the use of different concentrations of recombinant human ANGPTL4 fragments. The monoclonal antibody (ED12B9) was used as a coating antibody (5 μg/ml) and the biotinylated rabbit anti-human ANGPTL4 IgG was used as a detection antibody (0.6 μg/ml). The intra- and inter-assay coefficients of variance (CV) were 2.9-6.8% and 3.7-6.1% respectively, and the linear range of the assay was 0.5-50 ng/ml. Note that this assay has no cross-reactivity with the major components of human serum (albumin and immunoglobins) or several other adipokines in the circulation (adiponectin, resistin and leptin).

FIG. 6 shows that serum levels of ANGPTL4 are decreased in patients with T2DM, but not in obese individuals without hyperglycemia. [A] Comparison of serum ANGPTL4 concentrations between patients with T2DM and age-, BMI- and sex-matched healthy individuals; *, P<0.05; **, P<0.01 B: Comparison of plasma ANGPTL4 levels between healthy lean individuals (BMI<25), obese individuals without diabetes and obese individuals with diabetes (BMI>30). **, P<0.01 compared with lean healthy subjects or obese individuals without T2DM.

FIG. 7 shows that correlations between serum ANGPTL4 levels and plasma concentrations of glucose (A), HOMA IR (B), serum concentrations of triglycerides (C), and total cholesterol (D).

### DETAILED DESCRIPTION OF THE PREFERRED EMBODIMENTS

The invention provides a method for decreasing blood glucose or improving insulin sensitivity in a mammal in need thereof, comprising administering to the patient an amount of angiopoietin like protein-4 (ANGPTL4) polypeptide effective to decrease blood glucose, to improve glucose tolerance, or to increase insulin sensitivity. The mammal may be afflicted with diabetes, hyperglycemia, or other metabolic disorder or disease. The metabolic disorder is a metabolic syndrome associated with insulin resistance, Type 1 or 2 diabetes mellitus, polycystic ovary syndrome. The ANGPTL4 polypeptide is administered in a subcutaneous, intramuscular, intraperitoneal, or intravenous injection.

The ANGPTL4 polypeptide is purified from animal tissues or derived from a genetically engineered cell. The ANGPTL4 polypeptide preferably further comprises at least one control sequence operatively linked to the ANGPTL4 polypeptide, and at least one control sequence, which may be CMV, IE, SV40, RSV, LTR or a beta actin promoter.

Preferably, the polynucleotide encoding the ANGPTL4 polypeptide is inserted into a vector. The vector may be a viral or plasmid vector, such as an adenovirus, retrovirus, lentivirus, adeno-associated virus or herpes virus viral vector. The vector including the polynucleotide encoding the ANGPTL4 polypeptide is administered to a patient by subcutaneous, intramuscular, intraperitoneal, or intravenous injection.

The invention further provides a method for diagnosing diabetes and insulin resistance in an individual comprising obtaining a blood sample from a patient, and determining the amount of ANGPTL4 contained in the sample, comparing that amount against a reference number to determine whether the amount of ANGPTL4 polypeptide in that individual indicates the presence or absence of diabetes or insulin resistance. The amount of ANGPTL4 in the blood sample is measured using a sandwich immunoassay or a radioimmunoassay. An amount of ANGPTL4 lower than the reference level indicates the presence of diabetes or insulin resistance.

The invention also provides a method for monitoring a treatment for diabetes or insulin resistance in a patient comprising monitoring blood levels of ANGPTL4 polypeptide in the patient receiving treatment.

The invention additionally provides a method of screening for an agent useful for treatment of diabetes or insulin resistance comprising contacting a mammalian cell expressing an ANGPTL4 polypeptide with an agent, and determining if the agent increases ANGPTL4 polypeptide production. An increase in ANGPTL4 polypeptide production is indicative of usefulness for treatment of diabetes and insulin resistance. The mammalian cell may be from a rat, mouse, or human, and the cell is an adipocyte or a hepatocyte. The method may be carried out by administering a test agent to a mammalian subject. For example, the mammalian subject is a rodent having genetic or experimentally induced diabetes or insulin resistance.

The invention also provides a method of treating diabetes and insulin resistance in an individual comprising administering an agent that increases the endogenous production of AGPTL4 in the individual.

The following experimental section is illustrative of the practice of the invention, and is not meant to limit the scope of the invention in any way.

### Experimental Section

We employed an adenovirus-mediated expression system to investigate the metabolic effects of ANGPTL4 in mice. Our results demonstrated that ANGPTL4 is an important regulator of glucose homeostasis as well as insulin sensitivity. In both C57 mice and db/db diabetic mice, ANGPTL4 markedly improved glucose tolerance and decreased blood glucose, possibly by inhibition of hepatic glucose production. Moreover, our clinical study demonstrated that serum ANGPTL4 levels are inversely correlated with plasma glucose concentrations in human subjects, and are significantly decreased in patients with Type 2 Diabetes Mellitus (T2DM).

Methods

Animals. Male C57BU6J and C57BKS db/db diabetic mice (Jackson laboratory) between 8-10 weeks old were used for this study. The mice were housed in a room under controlled temperature (23±1° C.), with free access to water and standard mouse chow. All the experiments were conducted under our institutional guidelines for the humane treatment of laboratory animals.

Human clinical study protocol. A total of 42 lean healthy subjects, 46 patients with T2DM, and 22 obese individuals without T2DM were recruited for this study. The clinical characteristics of the subjects are given in Table 1:

TABLE 1
Clinical characteristics of the subjects recruited for this study.

| | | Obese without | |
|---|---|---|---|
| | Healthy Lean | T2DM | T2DM |
| Number (M/F) | 42(18/24) | 22(10/12) | 46(22/24) |
| Age | 41 ± 1 | 39 ± 2 | 48 ± 2 |
| BMI (kg/m²) | 21.0 ± 0.2 | **33.2 ± 1.2 | **39.5 ± 1.4 |
| Fasting glucose (mM) | 4.94 ± 0.05 | 5.15 ± 0.15 | **12.7 ± 0.72 |

*P < 0.05;
**P < 0.01 compared with healthy lean subjects

Fasting (12 h overnight) blood was taken for measurement of fasting plasma glucose (FPG), insulin, and total cholesterol and triglyceride as we previously described (14). The homeostasis model assessment of insulin resistance (HOMA IR), a simple assessment of insulin sensitivity, was calculated using the formula: FPG (mmol/l)×fasting insulin (μU/ml)/22.5. The study protocol was approved by the Ethics Committee of the Medical Faculty, the University of Hong Kong.
Cloning of mouse ANGPTL4 and construction of its mammalian expression vectors. Total RNA was obtained from 3T3-L1 adipocytes using Trizol reagent (Invitrogen) according to the manufacturer's instructions. Reverse transcription-PCR was performed based on the mouse ANGPTL4 nucleotide sequence (GenBank™/EBI accession number NM 020581), using 5′-GCCCGCGGATCCATGCGCTGCGCTCCGAC-3′ as the sense primer and 5′-GGCCGCGAATTCTCACTTGTCATCGTCGTCCTTGTAGTCAGAGGCTGCTGTAGC CTC-3′ as the anti-sense primer respectively. The amplified DNA fragment was digested with BamHI/EcoRI, and then inserted into pcDNA3.1(+) to produce the mammalian expression vector pcDNA-ANGPTL4-F, which encodes full-length ANGPTL4 with a FLAG epitope tag at its COOH terminus. The inserted ANGPTL4 cDNA was verified by DNA sequencing.
Expression and purification of human ANGPTL4 fragments from E. coli and production of antibodies. A 468-bp cDNA encoding a human ANGPTL4 fragment (amino acid residues 26-178) was sub-cloned into pROEX-HTb vector, which was then used to transform host E. coli, BL21 cells. The expression was induced by the addition of 1 mM of isopropylthio-β-D-galactoside (IPTG). His-tagged ANGPTL4 fragment was purified from the bacterial lysates using Ni²⁺-nitrilotriacetic acid-agarose column as we previously described (19). The purity of the protein was confirmed by SDS-PAGE and HPLC.

The monoclonal antibody against human ANGPTL4 was generated by immunization of female BALB/c mice with the recombinant human ANGPTL4 fragments until the polyclonal sera from mice exhibited strong immune responses. Splenocytes were then isolated from the mice and fused with the myeloma cells sp2/0. The positive clone (ED12B9) was selected and injected into the immunocompromised BALB/c mice, and the immunoglobulin was purified from ascites using the protein G-coupled column. The polyclonal antibody against the recombinant human ANGPTL4 fragment was raised in female New Zealand white rabbits as we previously described (19). The specificity of the antibodies was verified by Western blot.

Construction of adenoviral vector for expression of ANGPTL4, and production of adenoviruses. The adenovirus expression vector that encodes FLAG-tagged ANGPTL4 was generated using the Adeno-X Expression System (BD Biosciences, Clontech, Palo Alto, Calif.). The recombinant virus was packaged and amplified in HEK293 cells, and purified by CsCl density gradient centrifugation. The recombinant adenovirus that encodes luciferase was kindly provided by Dr. Christopher Rhodes (15).

Development of a Sandwich Enzyme-Linked Immunoassay for Measurement of Human ANGPTL4.

The anti-human ANGPTL4 polyclonal antibody was biotinylated with a kit from Pierce Chemical Co. and free biotin was removed by dialysis. The monoclonal antibody ED12B9 (5 μg/ml) was used for coating a 96-well microtiter plate overnight at 4° C. The coated plate was washed three times with PBS, and blocked with 100 μl of PBS containing 1% BSA and 0.05% Tween-20 for 2 hr. Human serum was diluted 1:50, and 100 μl of the diluted samples or standard were applied to each well, incubated at 37° C. for 1 hr, washed three times, then incubated with 100 μl of biotinylated anti-human ANGPTL4 polyclonal antibody for another 2 hr. Following three washes, the wells were incubated with streptavidin-conjugated horseradish peroxidase for 1 hr and subsequently reacted with tetramethyl-benzidine reagent for 15 min. 100 \i\ of 2M H2SO4 was added to each well to stop the reaction, and the absorbance at 450 nm was measured. The intra- and inter-assay coefficients of variance (CV) were determined by measuring five serum samples from healthy subjects in a total of six independent assays with duplicate determinations.

Glucose tolerance test (GTT). Mice were placed in clean cages with no food but with free access to water at ˜9:00 a.m. After a 6-h starvation, mice were weighed, and the tip of the tail was clipped to obtain blood for glucose measurement. Mice were injected intraperitoneally with glucose (1 g/kg body weight). Blood (˜5 μl) was taken from the tail tip at various time points for measurement of glucose concentration, using a Glucose meter Elite (Bayer, Leverkusen, Germany).

Analysis of insulin and adiponectin levels in mouse serum. Circulating concentrations of mouse insulin were quantified using the commercial ELISA kits from Mercodia AB (Uppsala, Sweden). Circulating adiponectin was determined using an in house ELISA established previously in our laboratory (16).

Oil Red O staining of liver sections and quantification of hepatic glycogen contents. Oil Red O staining of lipid droplets in liver sections was performed as we previously described (17). The glycogen content in the liver extracts of control and transgenic mice was determined with amyloglucosidase according to the method of Keppler and Decker (18).

Isolation of primary rat hepatocytes and measurement of hepatic glucose output. Primary hepatocytes were prepared from male Wistar rats (˜200 g) as we previously described (19). Cells were plated on collagen type I-coated 12-well plates in DMEM with 10% fetal bovine serum, 2 mM L-glutamine, 10 μM dexamethasone, and 10 μg/ml insulin at a density of 5×10⁵ cells/well. The cells were allowed to adhere to the cell culture dishes for 8 h and then infected with Adv-ANGPTL4 or Adv-Luc at the concentrations of 50 p.f.u/cell. At 24 hr after infection, the cells were stimulated without or with different concentrations of insulin for another 24 hr. The medium was then replaced with 0.5 ml of glucose-free DMEM without phenol red and supplemented with 5 mM each alanine, valine, glycine, pyruvate, and lactate. After incubation for 6 h, the glucose level in the medium was measured using the glucose Trinder assay kit (Sigma-Aldrich).

Statistical analysis. Experiments were performed routinely with five to six mice per group with values presented as mean plus or minus SE. All the studies were replicated with representative data shown. Statistical significance was determined by one-way ANOVA. In all statistical comparisons, a P value of less than 0.05 was used to indicate a significant difference.

Result

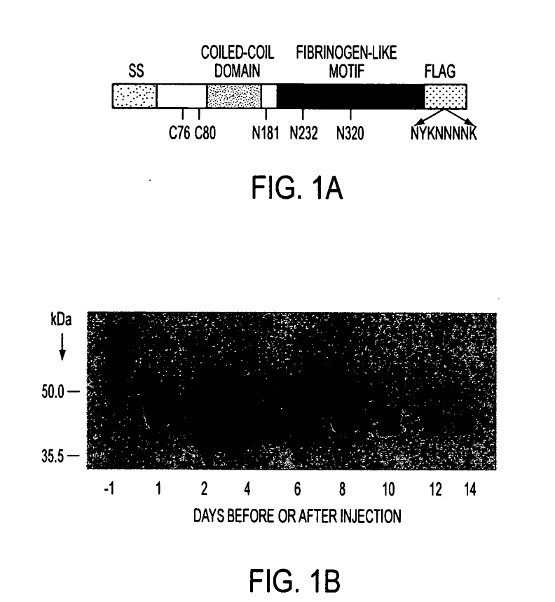

ANGPTL4 potently decreases blood glucose levels and improves glucose tolerance in C57 mice. To investigate the metabolic functions of ANGPTL4 in vivo, we generated the recombinant adenovirus that encodes the mouse full-length ANGPTL4 tagged with the FLAG-epitope to facilitate the detection of the expressed protein. 5×10⁹ p.f.u of the recombinant adenovirus that expresses ANGPTL4 (Adv-ANGPTL4), or luciferase (Adv-Luc as a control) was introduced into C57 mice through tail vein injection. Expression of FLAG-tagged ANGPTL4 protein was detected in the circulation at day 1, peaked at day 4, and subsequently attenuated (FIG. 1). A trace amount of ANGPTL4 expression was still detectable at 2 weeks after injection. The apparent molecular weight of ANGPTL4 detected in the circulation is ˜50 kDa, which is equivalent to the carboxyl terminus of its proteolytic products (20).

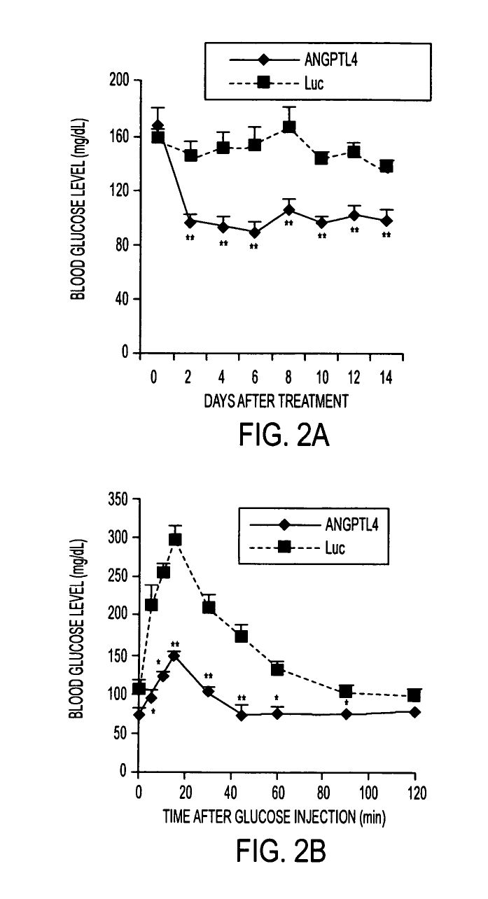

ANGPTL4 over-expression markedly decreased blood glucose levels in both fasted and ad libitum states, as shown in FIG. 2A and Table 2 below.

TABLE 2
Hepatic glycogen contents and plasma levels of glucose,
FFA, insulin and adiponectin in mice after treatment
with Adv-ANGPTL4 or Adv-Luc for two weeks.

| | Adv-Luc | Adv-ANGPTL4 |
|---|---|---|
| Fed glucose (mg/L) | 141.2 ± 8.7 | 98.2 ± 4.3** |
| Fasting glucose (mg/L) | 113.8 ± 6.1 | 82.7 ± 3.6** |
| Free fatty acid (mM) | 0.459 ± 0.063 | 0.628 ± 0.118* |
| Hepatic glycogen (mg/g liver tissue) | 3.22 ± 0.24 | 5.90 ± 0.53** |
| Insulin (μg/L) | 0.534 ± 0.06 | 0.599 ± 0.08 |
| Adiponectin (μg/ml) | 8.12 ± 0.77 | 8.33 ± 0.56 |

*P < 0.05;
**P < 0.01 compared with mice treated with Adv-Luc.

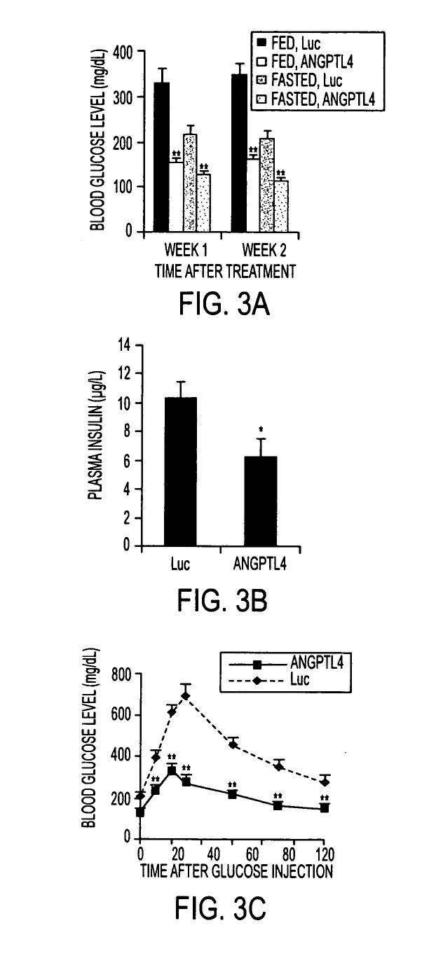

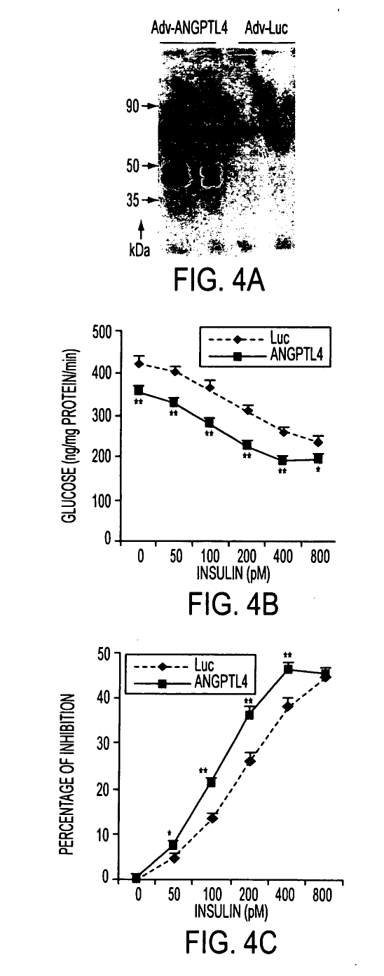

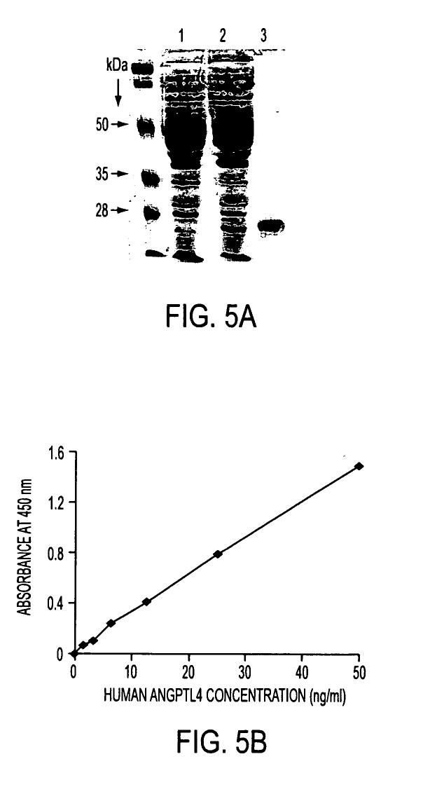

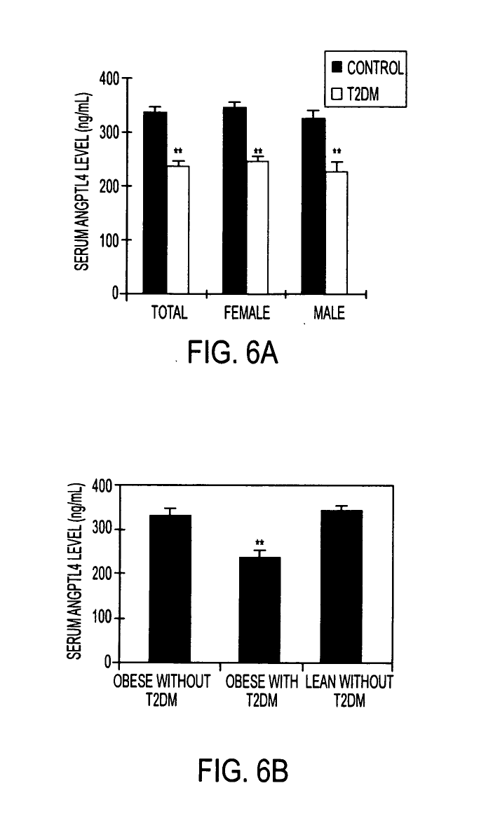

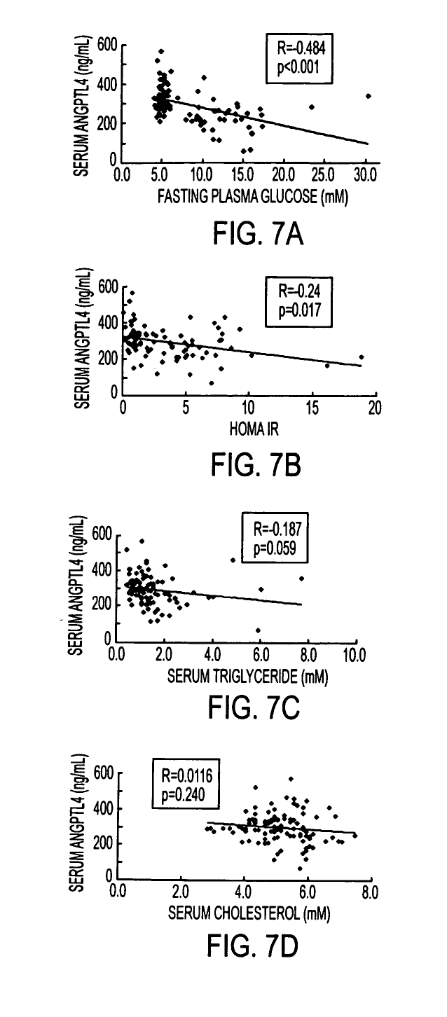

The glucose-lowering effect of ANGPTL4 was observed at day 2 after injection with Adv-ANGPTL4, and sustained throughout the treatment period. Two weeks after injection, blood glucose levels in mice injected with Adv-ANGPTL4 were still significantly lower than that in control mice injected with Adv-Luc (see Table 2). Hepatic glycogen content was significantly higher in Adv-ANGPTL4 treated group than that in controls, indicating that ANGPTL4 might enhance insulin sensitivity in the liver tissue. Notably, when challenged with an i.p. glucose load, the mice treated with Adv-ANGPTL4 showed a much lower peak glucose concentration at 15 min, and a faster decline of blood glucose levels throughout the glucose tolerance curve, suggesting that ANGPTL4 might also increase peripheral glucose disposal (FIG. 2B). ANGPTL4 treatment did not significantly affect the plasma concentrations of insulin or adiponectin in C57 mice.
ANGPTL4 treatment markedly alleviates hyperglycemia, hyperinsulinemia, and glucose intolerance associated with db/db diabetic mice. We next investigated the effect of ANGPTL4 on energy metabolism and insulin sensitivity in C57BLKS db/db mice, a genetically inherited diabetic mouse model that is characterized by severe hyperglycemia, hyperinsulinemia and glucose intolerance. The changes of lipid profiles in mice injected with Adv-ANGPLTL4 were similar to those in C57 lean mice. The serum levels of both triglycerides and cholesterol increased sharply for the first 6 days after injection, and this effect became attenuated afterwards (data not shown). On the other hand, hyperglycemia in both fasted and ad libitum states was sharply decreased to a normal level in db/db mice injected with Adv-ANGPTL4 (FIG. 3A). This potent glucose-lowering effect was observed at day 2 after injection with Adv-ANGPTL4, and sustained for at least two weeks. In addition, ANGPTL4 treatment significantly decreased hyperinsulinemia and markedly improved glucose intolerance associated with this diabetic mouse model (FIGS. 3B and 3C). A similar glucose-lowering, hypoinsulinemic and insulin-sensitizing effect was also observed in high fat diet-fed C57 mice with insulin resistance (data not shown).
ANGPTL4 inhibits glucose production in rat primary hepatocytes. Our finding that ANGPTL4 can decrease blood glucose and increase hepatic glycogen contents suggests that the liver might be the target tissue of this protein. We next investigated the direct effect of ANGPTL4 on glucose output of rat primary hepatocytes by infecting these cells with Adv-ANGPTL4 or Adv-luc. ANGPTL4 protein in the culture media of rat primary hepatocytes was detected as the full-length as well as the proteolytic products equivalent to its carboxyl terminus (FIG. 4A). Notably, the basal hepatic glucose output (HGO), as determined by measurement of glucose contents released into culture media, was significantly decreased in hepatocytes treated with Adv-ANGPTL4 (FIG. 4B). In addition, ANGPTL4 treatment enhanced the sensitivity of insulin to inhibit hepatic glucose output in this system (FIG. 4C), suggesting that inhibition of HGO might represent a potential mechanism underlying the glucose-lowering effect of ANGPTL4.
Serum concentrations of ANGPTL4 are decreased in patients with T2DM and are inversely correlated with plasma glucose levels and HOMAIR. To validate the role of ANGPTL4 as a circulating hormone in humans, we established a sandwich ELISA method for measurement of this protein in human plasma. To this end, we generated both the monoclonal and polyclonal antibodies against human ANGPTL4, using a recombinant human ANGPTL4 fragment as an antigen (see FIG. 5A). Western blot analysis revealed that both antibodies specifically recognize the recombinant human ANGPTL4 fragments (data not shown). The specificity of the anti-human ANGPTL4 monoclonal and polyclonal antibodies was further validated by the fact both these antibodies can specifically immunoprecipitate the recombinant human ANGPTL4 from bacterial lysates (data not shown). The sandwich ELISA standard curve (FIG. 5B) using the human recombinant ANGPTL4 fragments yielded a consistent r² value greater than 0.985.
Serum ANGPTL4 concentrations of healthy individuals were 345.04±10.83 ng/ml in both genders. Notably, its serum concentrations in patients with T2DM were substantially lower than in non-diabetic subjects, and this difference was significant in each gender group (FIG. 6A). On the other hand, serum concentrations of ANGPTL4 in obese individuals without T2DM were similar to those in non-obese individuals (FIG. 6B). Bivariate correlation analysis showed a significant inverse relationship between the serum levels of ANGPTL4 and plasma glucose concentrations as well as HOMAIR (FIG. 7), suggesting that ANGPLT4 might also act as a glucose-lowering and insulin-sensitizing hormone in humans. On the other hand, there were no significant correlation between serum ANGPTL4 concentrations and serum levels of triglycerides and total cholesterol.
Discussion

ANGPTL4 is a downstream target gene of both PPARγ and PPARα, the agonists of which are widely used for the treatment of type 2 diabetes, insulin resistance and dyslipidemia. In this study, we provided direct evidence that ANGPTL4 is a blood-borne hormone involved in regulating glucose homeostasis, lipid metabolism, and systemic insulin sensitivity. Over-expression of ANGPTL4 in mice potently decreased blood glucose levels and improved glucose tolerance. In db/db diabetic mice, ANGPTL4 treatment reduced hyperglycemia to a normal level, and markedly alleviated glucose intolerance and hyperinsulinemia. Notably, chronic treatment of db/db mice with PPARγ agonists also produced a similar insulin-sensitizing and glucose-lowering effect (21, 22), suggesting that the anti-diabetic actions of PPARγ agonists might be partly mediated by induction of ANGPTL4 production.

Our results suggest that the glucose-lowering effect of ANGPTL4 could be due to its direct actions on hepatocytes. This conclusion is supported by the fact that ANGPTL4 suppresses basal glucose output and enhances the sensitivity of insulin to inhibit glucose production in primary rat hepatocytes. The inhibitory effect of ANGPTL4 on hepatic glucose production is reminiscent of adiponectin (19, 23), another adipokine induced by the PPARγ agonists. However, unlike ANGPTL4, adiponectin has no effect on basal glucose output. Adiponectin exerts its hepatic actions by activation of AMP-activated protein kinase (24), whereas we found that ANGPTL4 had no effect on this kinase in mice (data not shown), suggesting that these two adipokines might act through distinct pathways. The detailed metabolic pathways and signal transduction events that underlie the hepatic action of ANGPTL4 on glucose metabolism remain to be clarified.

The role of ANGPTL4 as a potential glucose-lowering hormone was also supported by our finding that serum levels of ANGPTL4 are inversely correlated with plasma glucose concentrations in human subjects. Furthermore, the serum ANGPTL4 levels in patients with T2DM, but not in obese subjects without diabetes, are substantially decreased, suggesting that decreased ANGPTL4 could be a causative factor of hyperglycemia. It is interesting to note that adiponectin, another fat-derived hormone with direct insulin sensitizing and glucose-lowering functions, is also decreased in T2DM patients (25, 26). However, unlike adiponectin, serum ANGPTL4 levels lack obvious correlation with adiposity, and the circulating concentrations of triglycerides and total cholesterol, suggesting that lipid-elevating effects of ANGPTL4 observed in mice might not be physiologically relevant.

In plasma, ANGPTL4 is present as a full-length oligomerized protein as well as the proteolytically cleaved form (11, 20). The processing of ANGPTL4 oligomerization and proteolysis is similar with that of adiponectin. In the case of adiponectin, its proteolytic fragment is functionally different from the full-length adiponectin, with the former enhancing muscular fatty acid oxidation (5, 6) and the later inhibiting hepatic glucose production (19, 23). Different oligomeric forms of adiponectin have also been reported to act on different target tissues and activate distinct signaling pathways (27). Given the similarities between adiponectin and ANGPTL4, it is tempting to speculate that the multiple metabolic effects of ANGPLT4 on glucose and lipid metabolism might be differentially mediated by its distinct oligomeric complexes or proteolytic fragments.

In summary, our present study provided both clinical and experimental evidence supporting the role of ANGPTL4 as a blood-borne hormone involved in maintaining glucose homeostasis. The glucose-lowering effect of ANGPTL4 might be partly mediated by its direct inhibition on hepatic glucose output. We believe that further elucidation of molecular and structural mechanism that underlie the multiple metabolic effects of ANGPTL4 might help to develop novel therapeutic strategies for T2DM and other obesity-related metabolic disorders.

### REFERENCES

1. Kershaw, E. E. and Flier, J. S., J. Clin. Endocrinol. Metab., 89, 2548-56 (2004).
2. Lyon, C. J. et al., Endocrinology, 144, 2195-200 (2003).
3. Friedman, J. M. & Halaas, J. L. Nature, 395, 763-70 (1998).
4. Combs, T. P. et al., J. Clin. Invest., 108, 1875-81 (2001).
5. Yamauchi, T. et al., Nat. Med., 7, 941-6 (2001).
6. Fruebis, J. et al., Proc. Natl. Acad. Sci. U.S.A., 98, 2005-10 (2001).
7. Matsuda, M. et al., J. Biol. Chem., 277, 37487-91 (2002).
8. Yoon, J. C. et al., Mol. Cell. Biol., 20, 5343-9 (2000).
9. Kersten, S. et al., J. Biol. Chem., 275, 28488-93 (2000).
10. Kim, I. et al., Biochem. J., 346, 603-10 (2000).
11. Mandard, S. et al., J. Biol. Chem., 279, 34411-20 (2004). Epub 2004 Jun. 9.
12. Yoshida, K. et al., J. Lipid Res., 43, 1770-2 (2002).
13. Ge, H. et al., J. Lipid Res., 45, 2071-9 (2004). Epub 2004 Aug. 1.
14. Wat, N. M. et al., Int. J. Obes. Relat. Metab. Disord., 25, 1789-93 (2001).
15. Wrede, C. E. et al., J. Biol. Chem., 277, 49676-84 (2002). Epub 2002 Oct. 21.
16. Xu, A. et al., Endocrinology, 145, 487-494 (2004).
17. Xu, A. et al., J. Clin. Invest, 112, 91-100 (2003).
18. Keppler, D. and Decker, K., In Methods of Enzymatic Analysis, Vol. VI., Bergmeyer NU, Ed. Weinheim, Germany, VCH Publishers, 2225-2228 (1974).
19. Wang, Y. et al., J. Biol. Chem., 277, 19521-9(2002).
20. Ge, H. et al., J. Biol. Chem., 279, 2038-45 (2004). Epub 2003 Oct. 21.
21. Berger, J. et al., J. Biol. Chem., 274, 6718-25 (1999).
22. Berger, J. et al., Endocrinology, 137, 4189-95 (1996).
23. Berg, A. H. et al., Nat. Med., 7, 947-53 (2001).
24. Yamauchi, T., et al., Nat. Med. 8, 1288-95 (2002).
25. Hofta, K. et al., Arterioscler. Thromb. Vasc. Biol., 20, 1595-9 (2000).
26. Stefan, N. et al., Diabetes, 51, 1884-8 (2002).
27. Tsao, T. S. et al., J. Biol. Chem., 278, 50810-50817 (2003).

We claim:

1. A method for decreasing blood glucose, improving glucose tolerance, or increasing insulin sensitivity, in a mammal in need thereof, comprising administering to the patient an amount of angiopoietin like protein-4 (ANGPTL4) polypeptide effective to decrease blood glucose, to improve glucose tolerance, or to increase insulin sensitivity.

2. A method in accordance with claim 1, wherein the mammal is afflicted with diabetes, hyperglycemia, or a metabolic syndrome associated with insulin resistance, Type 1 or 2 diabetes mellitus, or polycystic ovary syndrome.

3. A method in accordance with claim 1, wherein the ANGPTL4 polypeptide is administered in a subcutaneous, intramuscular, intraperitoneal, or intravenous injection.

4. A method in accordance with claim 2, wherein the AGPTL4 polypeptide is purified from animal tissue or derived from a genetically engineered cell.

5. A method in accordance with claim 1, wherein the ANGPTL4 polypeptide further comprises at least one control sequence operatively linked to the ANGPTL4 polypeptide.

6. A method in accordance with claim 5, wherein the at least one control sequence is a CMV, IE, SV40, RSV, LTR or beta actin promoter.

7. A method in accordance with claim 6, wherein the polynucleotide encoding the ANGPTL4 polypeptide is inserted into a vector.

8. A method in accordance with claim 7, wherein the vector is a viral or plasmid vector.

9. A method in accordance with claim 7, wherein the vector is an adenovirus, retrovirus, lentivirus, adeno-associated virus or herpes virus viral vector.

10. A method in accordance with claim 7, wherein the vector including the polynucleotide encoding the ANGPTL4 polypeptide is administered to a patient by subcutaneous, intramuscular, intraperitoneal, or intravenous injection.

11. A method for diagnosing diabetes or insulin resistance in an individual comprising obtaining a blood sample from a patient, and determining the amount of AGPTL4 contained in the sample, comparing that amount against a reference number to determine whether the amount of ANGPTL4 polypeptide in that individual indicates the presence or absence of diabetes or insulin resistance.

12. A method in accordance with claim 11, wherein an amount of ANGPTL4 lower than the reference level indicates the presence of diabetes or insulin resistance.

13. A method in accordance with claim 12, wherein the amount of ANGPTL4 in the blood sample is measured using a sandwich immunoassay or a radioimmunoassay.

14. A method for monitoring a treatment for diabetes or insulin resistance in a patient comprising monitoring blood levels of ANGPTL4 polypeptide in the patient receiving treatment.

15. A method of screening for an agent to determine its usefulness for treatment of diabetes or insulin resistance comprising contacting a mammalian cell expressing a ANGPTL4 polypeptide with the agent, and determining if the agent increases AGPTL4 polypeptide production, wherein an increase in ANGPTL4 polypeptide production is indicative of usefulness for treatment of diabetes or insulin resistance.

16. A method in accordance with claim 15, wherein the mammalian cell is from a rat, mouse, or human.

17. A method in accordance with claim 16, wherein the mammalian cell is an adipocyte or a hepatocyte.

18. A method in accordance with claim 15, wherein contacting the mammalian cell occurs by administering a test agent to a mammalian subject.

19. A method in accordance with claim 18, wherein the mammalian subject is a rodent having genetic or experimentally induced diabetes or insulin resistance.

20. A method of treating diabetes or insulin resistance in an individual comprising administering an agent that increases the endogenous production of ANGPTL4 in the individual.


SEQUENCE LISTING (3 sequences)

SEQ ID NO: 1 - DNA, 29 bp, Artificial Sequence
  Mouse ANGPTL4 Sense Primer
gcccgcggatccatgcgctgcgctccgac

SEQ ID NO: 2 - DNA, 57 bp, Artificial Sequence
  Mouse ANGPTL4 Anti-Sense Primer
ggccgcgaattctcacttgtcatcgtcgtccttgtagtcagaggctgctgtagcctc

SEQ ID NO: 3 - protein, 8 aa, Artificial Sequence
  Bacteriophage FLAG Epitope
NYKNNNNK